(19) United States
(12) Patent Application Publication
(10) Pub. No.: US 2003/0149375 A1
(43) Pub. Date: Aug. 7, 2003

(54) Body fat determining device

(76) Inventor: Chih-Yuh Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/062,769

(22) Filed: Feb. 5, 2002

(51) Int. Cl. A61B 5/05

(52) U.S. Cl. 600/547

(57) ABSTRACT

A body fat determining device takes the form of a weight measuring device, and has two pairs of electrodes, a display, and setting buttons on the upper sides. Each of the electrode pairs comes into contact with one of the subject's sole when the subject stands on the device. The electrodes of the electrode pairs can form current paths with each other, and are also provided for measuring the impedance of the subject's body such that the impedance of the contact between the subject's soles and the electrodes is eliminated from calculation. In other words, the accuracy can't be affected by unwanted factors such as moistures, roughness, or area of the subject's skin in contact with the electrodes. The device can find data that are stored in the memory, and belong to the present subject for calculation according to the datum on weight; thus, every subject only has to input data about him at the first time he uses the device.

### BACKGROUND OF THE INVENTION

The present invention relates to a body fat determining device, more particularly a body fat determining device capable of accurately determining a body fat ratio with the outcome of calculation and measurement being not affected by the moisture, roughness, and area of the skin of the subject in contact with the device. In addition, the body fat determining device of the present invention is equipped with a weight measuring means, and can store data of many subjects which are necessary for calculation of a body fat ratio, for example, age, height, and sex, and, when a particular subject stands on the fat determining device for having the body fat ratio determined, can provide the corresponding data to the calculating means for calculation of the body fat ratio according to the weight of the particular subject.

Referring to FIG. 5, a conventional body fat determining device includes a first electrode pair 51 and a second electrode pair 52. The electrode pairs 51 and 52 are provided on a surface of the main body of the body fat determining device such that a subject can electrically come into contact therewith by the fingers on the soles. The first electrode pair 51 includes a first current path forming electrode 511, and a first measuring electrode 512, while the second electrode pair 52 includes a second current path forming electrode 521, and a second measuring electrode 522. When the subject's hands or soles electrically come into contact with the electrode pairs 51 and 52 respectively, the first and the second current path forming electrodes 511 and 521, and the subject's body forms a current loop. And, the measuring electrodes 512 and 522 are used for measuring the voltage. With the change of the voltage as time goes on, an impedance measuring means can determine the impedance, which is then sent to the CPU for calculation of a body fat ration.

However, it is found that the above conventional body fat determining device has a drawback that the data on a subject necessary for calculation of a body fat ratio, e.g. age, height, and sex, have to be input every time the device is used on the subject. And, the weight of the subject has to be measured with another weight measuring device before the body fat determining device is used, causing the user inconvenience.

Therefore, another body fat determining device is provided which is also equipped with a weight measuring means such that the device can automatically get the data on the weight of a subject when the subject stands on the same with his/her soles electrically coming into contact with the electrodes of the body fat determining device. So, it is more convenient to use than the first conventional body fat determining device. However, the other data on a subject necessary for calculation of a body fat ratio such as the height and the sex still have to be input manually every time the device is used on the subject.

Furthermore, body of the above conventional body fat determining device have a common drawback that the accuracy of the outcome of measurement would be remarkably affected by the moisture, roughness, and area of the subjects skin in contact with the electrodes because the impedance is measured with two current path forming electrodes and two measuring electrodes. Consequently, the body fat ratio determined by the conventional device would be very different from the real one.

### SUMMARY OF THE INVENTION

Therefore, it is a main object of the present invention to provide a body fat determining device which is equipped with a weight measuring means, and can store data of many subjects that are necessary for calculation of a body fat ratio, and will provide the data of each of the subjects to the CPU when the subject stands on the fat determining device for having the body at ratio determined.

It is another object of the present invention to provide a body fat determining device, of which the accuracy will not be affected by the moisture, roughness or area of the subject's skin in contact with the electrodes thereof

The body fat determining device of the present invention includes a main body, a setting buttons, a pair of adjustment buttons, a CPU, a memory, two electrode pairs, a display, and a weight measuring means. The main body having two foot supporting portions on the surface. The setting button and the adjustment buttons are provided for a user to input data about a subject that are necessary for calculation of a body fat ratio.

The electrode pairs each have two electrodes; the electrodes of each of the pairs are arranged on a front portion and a rear portion of one of the foot supporting portions respectively. The electrodes are each a current path forming electrode plus a measuring electrode so as to be able of form four current loops together with the subject's body; a first loop passes through a front portion of the left sole, the body, and a rear portion of the right sole of the subject and has a first impedance; a second loop passes through a rear portion of the left sole, the body, and a front portion of the right sole, and has a second impedance; a third loop passed through the front and the rear portions of the left sole, and has a third impedance; a fourth loop passes through the front and the rear portion of the right sole and has a fourth impedance; the impedance are obtained according to RC oscillating principle. One half of a resultant impedance of the first impedance plus the second impedance minus the third impedance minus the fourth impedance, i.e. the impedance of the subject's body, is used together with the data by the CPU for determining the body fat ratio; thus, the impedance of contacts between the soles and the electrodes is eliminated from the calculation. In other words, the accuracy can't be affected by the moisture, roughness or area of the subject's skin in contact with the electrodes.

The weight measuring means can measure a weight of the subject when he stands on the foot supporting portions such that the CPU can find out data necessary for calculation of a body fat ratio that belong to the present subject and are stored in the memory according to the datum on weight. Therefore, the subject only has to input the data at the first time he uses the present body fat determining device.

### BRIEF DESCRIPTION OF THE DRAWINGS

The present invention will be better understood by reference to the accompanying drawings, wherein:

FIG. 1 is a perspective view of the body ratio determining device of the present invention.

FIG. 2 is a block diagram of the present invention.

FIG. 3 is a view showing the current loops of the present invention.

FIG. 4 is a flow chart of using the body fat determining device according to the present invention.

FIG. 5 is a view showing the current loops of the conventional body fat determining device.

### DETAILED DESCRIPTION OF THE PREFERRED EMBODIMENT

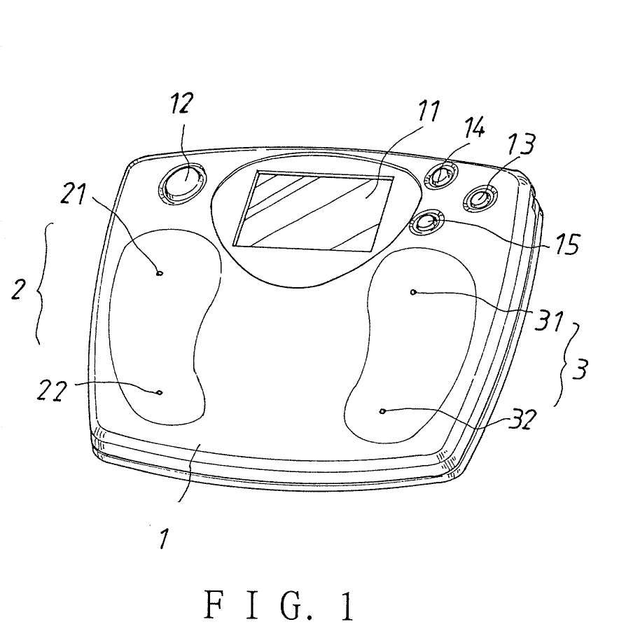

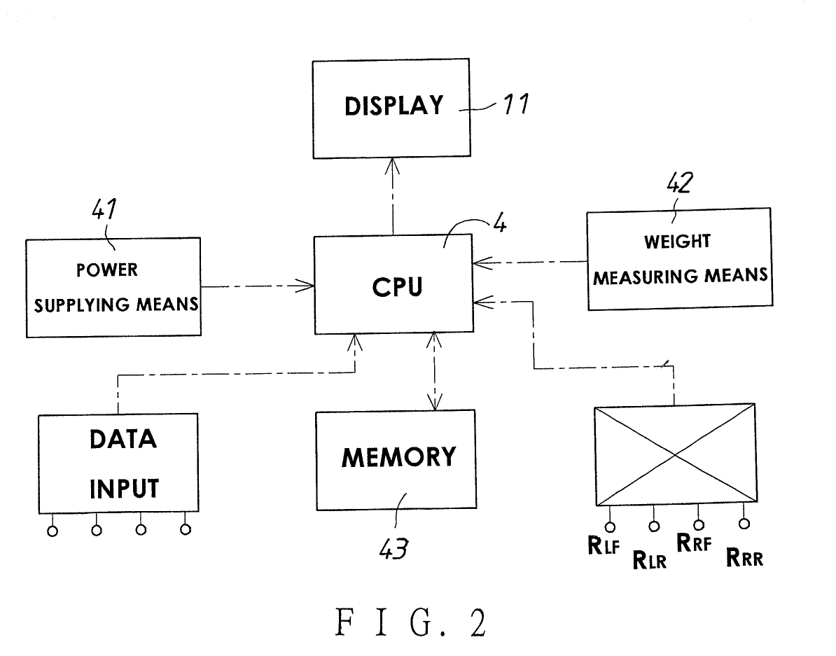

Referring to FIGS. 1, and 2, a body fat determining device of the present invention takes the form of a weight measuring device, and has a main body 1, which has a pair of foot supporting portions on the upper side for a subject to stand thereon. The body fat determining device includes a first electrode pair 2, a second electrode pair 3, a display 11, a switch 12, a setting button 13, and a pair of adjustment buttons 14, 15 on the upper side of the main body. The first and the second electrode pairs 2, 3 each includes an electrode 21(31) on a front portion of a corresponding one of the foot supporting portions, and an electrode 22 (32) on a rear portion of the corresponding foot supporting portion. Every one of the electrodes 21, 31, 22, and 32 is a current path forming electrode as well as a measuring electrode.

The body fat determining device further has a CPU 4 connected to the display 11, a power supplying means 41, a weight measuring means 42, and a memory 43. The power supplying means 41, the weight measuring means 42, and the memory 43 are all connected to the CPU 4. The electrodes 21, 31, 22 and 32 are also connected to the CUP 4.

The display 11 is provided for showing the body fat ratio of a subject, and the data necessary for calculation of a body fat ratio, e.g. a subject's age, height, and sex. The setting button 13, and the adjustment buttons 14, 15 are provided for a user to input the data necessary for calculation of a body fat ratio.

When a subject stands on the foot supporting portions of the main body 1, his/her soles will each electrically come into contact with one of the electrode pairs 2, 3. Thus, we can get impedance R_{LF}, R_{LR}, R_{RF}, R_{RR}, and R_{B} by means of finding out the oscillating frequency according to RC oscillating principle with the capacitance being fixed, wherein R_{LF} is the resultant impedance when the impedance R₁ of the front portion of the left sole is connected in series with the contact impedance r₁ between the electrode 21 and the front portion of the left sole; R_{LR} is the resultant impedance when the impedance R₂ of the rear portion of the left sole is connected in series with the contact impedance r₂ between the electrode 22 and the rear portion of the left sole; R_{RF} is the resultant impedance when the impedance R₃ of the front portion of the left sole is connected in series with the contact impedance r₃ between the electrode 31 and the front portion of the right sole; and, R_{RR} is the resultant impedance when the impedance R₄ of the rear portion of the right sole is connected in series with the contact impedance r₄ between the electrode 32 and the rear portion of the right sole. And, R_{B} is the impedance of the subject's body.

Because the subject's soles are in contact with the electrode pairs 2, 3, four current loops (1), (2), (3), and (4) are formed wherein current loop (1) passes through the electrodes 21, 22, and the front and the rear portions of the left sole; current loop (2) passes through the electrodes 31, 32, and the front and the rear portions of the right sole; current loop (3) passes through the electrodes 21, 32, the front portion of the left sole, the subject's body, and the rear portion of the right sole; and, current loop (4) passes the through the electrodes 22, 31, the rear portion of the left sole, the subject's body, and the front portion of the right sole. Therefore, the impedance of current loop (1) is R_{LF} plus R_{LR}; the impedance of current loop (2) is R_{RF} plus R_{RR}; the impedance of current loop (3) is R_{LF} plus R_{B} plus R_{RR}; and, the impedance of current loop (4) is R_{LR} plus R_{B} plus R_{RF}. And, we divide 2 into the resultant amount of the impedance of current loop (3) plus the impedance of current loop (4) minus the impedance of current loop (1) minus the impedance of current loop (2), we can get R_{B}, i.e. (R_{LF}+R_{B}+R_{RR}+R_{LR}+R_{B}+R_{RF}−R_{LF}−R_{LR}−R_{RF}−R_{RR}) /2=2R_{B}/2=R_{B}. Thus, the impedance of the contact between the soles and the electrodes is eliminated from the calculation of the CPU 4 for determining the fat ratio.

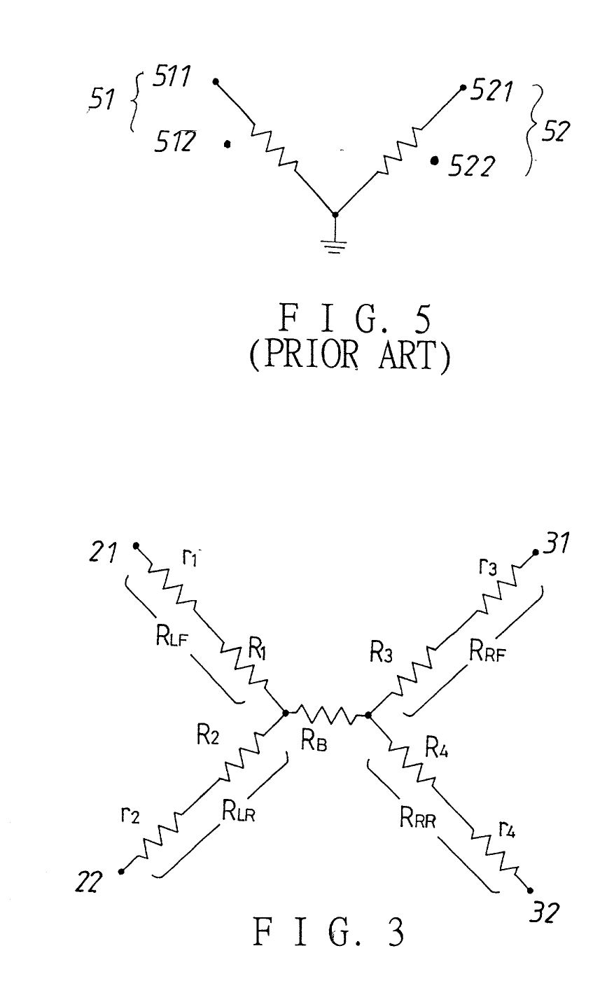

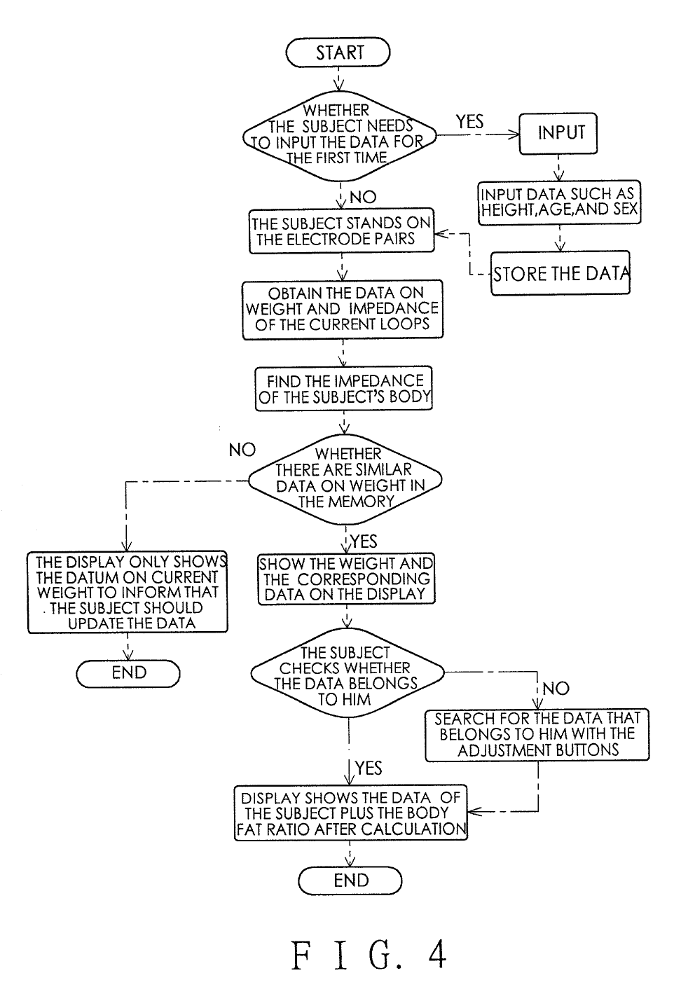

In using the body fat determining device of the present invention, referring to FIGS. 1 to 4, first the switch 12 is pushed so as to activate the power supplying means 41. If the present subject has never used this body fat determining device before, he/she should have the data input that are necessary for calculation of a body fat ration such as the height, age, and sex through the setting buttons 13, and the adjustment buttons 14, 15 Then, the subject stands on the foot supporting portions of the main body 1 to have the weight measured with the weight measuring means 42; thus, the weight is shown on the display 11, and put into the memory 43 and the CPU 4. The CPU will determine the body fat ration of the subject through calculation based on the above input data, and the impedance of the subject's body, R_{B}, which is found out through the electrodes 21, 22, 31 and 32. Finally, the body fat ratio plus the input data are shown on the display 11 for the subject.

If the present subject has used this body fat determining device before, he/she only has to stands on the foot supporting portions of the main body 1 to have his/her weight measured the CPU 4 will automatically search for the data necessary for the calculation of a body fat ratio that belong to him/her in the memory 43 according to the weight measured at the present time. Then, a body fat ratio is shown on the display 11 based on the data found by the CPU 4.

Because there will be some difference between the subject's weight of the last time and that of the present time, a certain tolerance is used such that the CPU 4 can find the data in the memory 43 that belong to the present subject providing that the difference is not greater than the tolerance.

The data found in the above mentioned way will also be shown on the display 11 for permitting the subject to check whether the data found by the CPU 4 belong to him/her. If the subject finds that the data shown on the display 11 don't belong to him/her, he/she will have to search for the correct data with the adjustment buttons 14, 15 such that the CPU 4 can determine the correct body fat ratio.

If the subject's weight has decreased or increased to such a degree that the difference between it and the weight input at the last time is greater than the tolerance, causing the CPU 4 to fail to find the data that belong to him/her, the display 11 will only show the subject's present weight in order to inform the subject that the weight data shown be updated.

From the above description, it can be easily understood that the body fat determining device of the present invention has the following advantages:

1. Because all the electrodes on the main body thereof are current path forming electrodes plus measuring electrodes, making four current loops formed between the electrodes, the CPU can eliminate those factors by means of calculation that would cause inaccuracy of the outcome, i.e. the impedance of the subject's body the factors can be moisture, roughness and area of the subject's skin in contact with the electrodes. In other words, the present body fat determining device can provide on accurate body fat ration.

2. The body fat determining device can store data necessary for calculation of a body fat ratio for the CPU to find. When a subject stands on the present device, the device can measure the weight of the subject, and the CPU can find all of the data that belong to the present subject in the memory according to the data on weight. Therefore, the user doesn't have to input data every time he/she wants to have the body fat ratio determined with the present device.

What is claimed is:

1. A body fat ratio determining device, comprising
a main body having two foot supporting portions thereon;
a setting button and a pair of adjustment buttons for permitting a user to input data about a subject that are necessary for calculation of a body fat ratio;
a display on said main body for showing a body fat ratio and said data;
a CPU for determining said body fat ratio through calculation based on said data plus a body impedance of the subject;
a memory connected to said CPU for storing said data;
two electrode pairs each having two electrodes; said electrodes of each of said pairs being arranged on a front portion, and a rear portion of one of said foot supporting portions respectively;
said electrodes being current path forming electrodes plus measuring electrodes so as to be able to form a first, a second, a third, and a fourth current loops together with the body of the subject standing on said foot supporting portions;
said first current loop passing through a front portion of a left sole, the body, and a rear portion of a right sole of the subject and having a first impedance; said second current loop passing through a rear portion of the left sole, the body, and a front portion of the right sole of the subject, and having a second impedance;
said third current loop passing through the front portion and rear portion of the left sole, and having a third impedance;
said fourth current loop passing through the front portion and the rear portion of the right sole, and having a fourth impedance;
said impedances being obtained by means of finding out oscillating frequency according to RC oscillating principle with the capacitance being fixed; said CPU using one half of a resultant impedance of said first impedance plus said second impedance minus said third impedance minus, which is an impedance of the subjects body, together with said data for determining a body fat ratio of the subject, thus eliminating impedance of contact between the soles and the electrodes from the calculation.

2. The body fat determining device as claimed in claim 1 further having a weight measuring means; said weight measuring means connected to said CPU;
said weight measuring means being capable of measuring a weight of a subject standing on said foot supporting portions such that said CPU can find out necessary data for calculation of a body fat ratio in said memory that belong to the subject according to an outcome of said weight measuring means measuring the subject's weight and a weight datum stored in said memory.
